(19) United States
(12) Patent Application Publication
(10) Pub. No.: US 2010/0173304 A1
(43) Pub. Date: Jul. 8, 2010

(54) METHOD OF DNA SEQUENCING

(75) Inventors: Ho-ming Pang, Ames, IA (US); Wei Wei, Ames, IA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: ADVANCED ANALYTICAL TECHNOLOGIES, INC., Ames, IA (US)

(21) Appl. No.: 12/646,270

(22) Filed: Dec. 23, 2009

Related U.S. Application Data
(60) Provisional application No. 61/140,218, filed on Dec. 23, 2008.

(51) Int. Cl. C12Q 1/68; B01D 57/02

(52) U.S. Cl. 435/6; 204/450

(57) ABSTRACT

The invention includes a method for sequencing DNA by partially sequencing the bases of complementary strands of double-stranded DNA and combining the partial information from both strands of the double strand DNA to fully sequence the DNA. The invention also includes DNA sequences sequenced by the methods, computer readable mediums including program instructions for such methods, and kits adapted to perform such methods.

### RELATED APPLICATIONS

This application claims the benefit of U.S. Provisional Application Ser. No. 61/140,218, titled Method of DNA Sequencing, filed Dec. 23, 2008, the contents of which are hereby incorporated by reference.

### FIELD OF THE INVENTION

In one aspect, the invention relates to a method of DNA sequencing. In another aspect, the invention relates to a DNA sequence determined, in whole or in part, by the use of the method described herein.

### BACKGROUND OF THE INVENTION

Deoxyribonucleic acid (DNA) is a long polymer with repeating units of nucleotides. The four nucleotide bases found in DNA are adenine (A), guanine (G), cytosine (C), and thymine (T). In living organisms, DNA exists as a tightly associated pair of polymers in the shape of a double helix. Each base on one strand of DNA is coupled through hydrogen bonding with one type of base on the other strand. The As and Ts are bonded together from across respective single strands in the double helix, thereby forming base pairs, as are the Gs and Cs. As a result of these known complementary associations, once the entire DNA sequence of one strand is known, the sequence of the other strand can be easily determined.

Maxam and Gilbert's DNA sequencing method by chemical degradation uses chemical reactions to cleave DNA at nucleotide-specific (to the particular base) sites. This reaction procedure can be used to determine the nucleotide sequence of a terminally labeled (e.g., with ³²P) DNA single strand by random breaking at adenine (A), guanine (G), cytosine (C), or thymine (T) positions using specific chemical agents. For each breakage, two fragments are generated from each strand of DNA. In order to perform a sequencing analysis, only one fragment has an associated label for later detection. A single reaction with the presence of formamide and piperidine can be used to cleave the phosphodiester bond at 3′ and 5′ positions. Instead of cutting only one nucleotide position, this method cleaves all nucleotides with relative efficiency A>G>C>T. Since there is higher cutting efficiency for A nucleotide compared to other nucleotides, the largest peaks on a representative electropherogram corresponds to base A. G has the second largest signal and the signal for C and T are proportionally smaller. The products of the original four cleavage reactions are separated by gel electrophoresis according to size (length), and autoradiographed. The pattern of bands on the x-ray film is read to determine the sequence of the original single strand DNA. From this, the sequence of the original complementary strand can be determined as well.

### SUMMARY OF THE INVENTION

Embodiments of the invention include a method for sequencing DNA that can include the use of as few as a single chemical cleavage reaction, while combining the information derived from both strands of an original dsDNA (double stranded DNA). Each strand of DNA in the dsDNA has a sequence complementary to each other, that is, when a DNA strand has the nucleotide adenine (A), the other strand will have the nucleotide thymine (T) in the corresponding position, and when one stand of DNA has guanine (G), the other strand will have cytosine (C). In accordance with embodiments of the invention, Applicant has discovered the manner in which each single strand of DNA can be used to provide partial sequence information, with the partial sequences of both single strands being combined along with the known complementary base pairs in order to determine the DNA sequence itself. In some embodiments, the partial sequence of each strand is determined with a single cleaving agent. In such embodiments, the cleaving step consists of cleaving the first and second strands with a single cleaving agent. In other embodiments, the partial sequence of each strand is determined with two cleaving agents. In such embodiments, the cleaving step consists of cleaving the first and second strands with two cleaving agents. In yet other embodiments, the partial sequence of each stand is determined with three cleaving agents. In such embodiments, the cleaving step consists of cleaving the first and second strands with three cleaving agents.

In one preferred embodiment, the respective strands of a dsDNA are initially labeled with different labels, e.g., one strand DNA is labeled at the 5′ end while other strand of DNA is labeled at the 3′ end, in order to permit fragments derived from either strand to be distinguished within a single sample. Thereafter, the strands can be subject to cleavage using one or more cleavage reactions having a desired and differential (e.g., “relative”) efficiency for cleavage as between the different bases. For instance, a single reactant can be used having essentially equal efficiency at cleaving all A and G residues, with lesser efficiency cleaving C residues, and essentially no ability to cleave T residues. Those skilled in the art will appreciate the manner in which, as described herein, the sequence of the original dsDNA can be determined once the corresponding fragments are separated and the two strands compared, knowing among other things, the relative cleavage efficiency of the original cleavage reactant(s).

Embodiments of the invention also include a DNA nucleotide sequence determined by the use of any of the methods described herein. In some embodiments, the invention includes a composition comprising fragments of dsDNA that has had its respective ssDNA strands differently labeled. Embodiments of the invention also include an electrophoretic gel comprising these DNA fragments separated according to size. Other embodiments of the invention include an isolated DNA sequence, or a degenerate variant thereof, comprising a sequence determined by any of the methods described herein. Embodiments of the invention also include a protein or polypeptide sequence corresponding to a nucleotide sequence determined by any of the methods described herein.

Other embodiments of the invention include a computer-readable medium including program instructions for performing the methods described herein. Embodiments of the invention also include a method comprising the use of such a computer-readable medium to perform the methods described herein.

Embodiments of the invention also include a kit consisting of a single, two, or three cleavage reagent(s), together with other ingredients and instructions for use in performing the methods described herein.

### DESCRIPTION OF THE DRAWINGS

FIG. 1 shows an electropherogram of one strand DNA fragments labeled with FAM at the 5′ position and chemically cleaved in accordance with an embodiment of the invention.

FIG. 2 shows an electropherogram of the complementary strand DNA fragments labeled with Cy-5 at the 3′ position and chemically cleaved in accordance with an embodiment of the invention.

FIG. 3 shows the overlay of electropherograms shown in FIG. 1 and FIG. 2 in accordance with an embodiment of the invention.

### DETAILED DESCRIPTION OF THE INVENTION

For the purpose of promoting an understanding of the principles of the invention, reference will now be made to embodiments of the invention, and specific language will be used to describe the same. It will, nevertheless, be understood that no limitation of the scope of the invention is thereby intended; any alterations and further modifications of the described or illustrated embodiments, and any further applications of the principles of the invention as illustrated therein, are contemplated as would normally occur to one skilled in the art to which the invention relates.

Embodiments of the invention include a method of sequencing DNA. By “sequencing DNA” it will be understood that the identity of at least two nucleotides in the DNA will be determined. The method includes the step of separating the strands (“unzipping”) of a double stranded DNA, having complementary base pairs, to provide a first single strand and a complementary second single stand. Double strand DNA can be readily separated into individual strands using a variety of techniques, e.g., by elevating the solution temperature to over 90° C. The first strand is labeled with a first label from a 5′ direction, and the second strand is labeled with a second label from a 3′ direction. For example, one strand of DNA could be labeled through a polymerase chain reaction (PCR) using dye labeled primer, while the other strand of DNA can be labeled using Klenow DNA polymerase. In general, it is preferred that the second label is differentially detectable from the first label. In some embodiments, the labeled first and second strand DNAs are cleaved with a single cleaving agent having a relative cleaving efficiency. The bases of both the first and second single strands are partially identified, and the sequence of the DNA is determined by combining the partial identification of the first single strand bases and the partial identification of the complementary second single strand bases.

In some embodiments, the single strands can be labeled by the use of any suitable labeling agent(s). For example, a fluorescently labeled primer can be used. In general, different labels are used for each respective single strand so it can be determined which base fragments came from which strand in the identification step. In some embodiments, a fluorescently labeled primer (e.g., at the 5′ position) as used in the course of PCR reactions can be used for one strand. In this way, one strand of DNA is labeled with fluorescent dye at the 5′ position. In addition, the other strand can be labeled at the 3′ terminus, e.g., using a polynucleotide kinase or Klenow DNA polymerase.

In some embodiments, one strand of DNA is only labeled at the 3′ position and the other complementary strand of DNA is only labeled at the 5′ position (e.g., using Klenow DNA polymerase). In such embodiments, the primer pair is selected in such a way that both primers do not have the same nucleotide at the 5′ position. For example, if one primer has a nucleotide A at the 5′ position, the other primer must have a nucleotide other than A at the 5′ position. This approach ensures that both DNA strands of the PCR product will have different nucleotides at the 3′ position. For example, if the 5′ fluorescent labeled strand DNA has an A nucleotide at the 3′ position, the other unlabeled strand DNA can have a C (G or T) nucleotide at the 3′ position. When Klenow DNA polymerase is used to replace the 3′ nucleotides, a fluorescent labeled C (G or T) nucleotide is added into the reaction solution with the Klenow DNA polymerase. This polymerase will replace the 3′ position C with fluorescently labeled C. After the reaction, one strand of DNA will have a fluorescent label at the 5′ position while other strand of DNA will have a different fluorescent label at the 3′ position. Accordingly, the base fragments from each strand may be identified and distinguished.

Any cleaving agent with a relative cleaving efficiency can be used. The four nucleotides may be generically referred to as nucleotides 1 through 4. In some embodiments of the invention, the relative cleaving efficiency of the cleaving agent is nucleotide 1>nucleotide 2>nucleotide 3>nucleotide 4 (i.e., the cleaving efficiency at nucleotide 1 is greater than at nucleotide 2, which is in turn greater than at nucleotide 3, which is in turn greater than at nucleotide 4). In yet other embodiments of the invention, the relative cleaving efficiency of the cleaving agent is nucleotide 1≈nucleotide 2>nucleotide 3, with insignificant cleaving efficiency for nucleotide 4 (i.e., the cleaving efficiency at nucleotide 1 is about the same as at nucleotide 2, which is in turn greater than at nucleotide 3). In yet other embodiments of the invention, the relative cleaving efficiency of the cleaving agent is nucleotide 122 nucleotide 2, with insignificant cleaving efficiency for nucleotides 3 and 4.

Specific examples of suitable cleaving agents include dimethyl sulfate and alkali, which can be used to cleave DNA at the G and A nucleotide positions with G having a cleaving efficiency of about five times higher than A. As another example, sodium hydroxide can be used to modify A and C and piperidine can be used to cleave the modified DNA at the A and C positions with A>C efficiency. As an additional example, methylamine can be used to modify G and T and UV irradiation can be used to cleave the modified DNA at the G and T positions with G>T efficiency. As another example, DNA could be cleaved with an 80% (w/w) solution of N-methylformamide at 110° C. with high cleaving efficiency at the G and A positions and moderate cleavage at the C positions and no significant cleavage at the T positions (A≈G>C). None of these chemical cleavage reactions provide enough information to determine the DNA sequence from a single reaction on a single strand. Using the information provided on a single strand of DNA typically requires multiple reactions, based upon the use of other nucleotide base cleaving agents, followed by and electrophoresis separation in order to generate enough information to determine the sequence.

Embodiments of the invention use both strands' DNA cleavage fragments to determine the DNA sequence. Any suitable method can be used to cleave and label the DNA. Using both strands' DNA fragment information provides complementary information to determine the full sequence of the DNA. After the cleavage on both strands of DNA, electrophoresis can be used to separate the DNA fragments according to their sizes. In some embodiments, fluorescent detection is used to monitor the signals associated with respective DNA fragments, and electropherograms of each strand may be created. Examples include electrophoresis instruments such as the DNA PROFiler or cePRO 9600 Fl, available from Advanced Analytical Technologies, Inc., Ames, Iowa, assignee of the present application. The electropherograms of each single strand are used together to determine the DNA sequence.

As an example, consider a hypothetical DNA with the following sequence:

*5′-TTCTGCAGTACACAAAATGCTCGTACACGACTATGACACGTACATC
ACCAGCGAATAGTTAATGGTA-3′

The other strand of DNA in the dsDNA has the following complementary sequence:

**3′-AAGACGTCATGTGTTTTACGAGCATGTGCTGATACTGTGCATGTA
GTGGTCGCTTATCAATTACCAT-5′

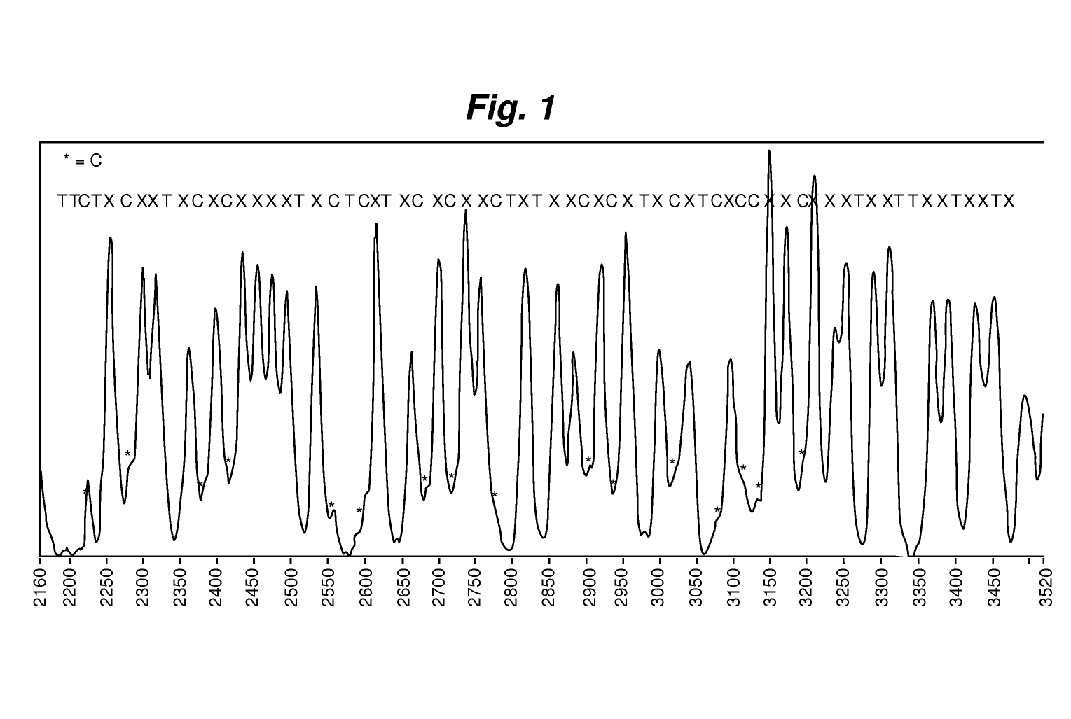

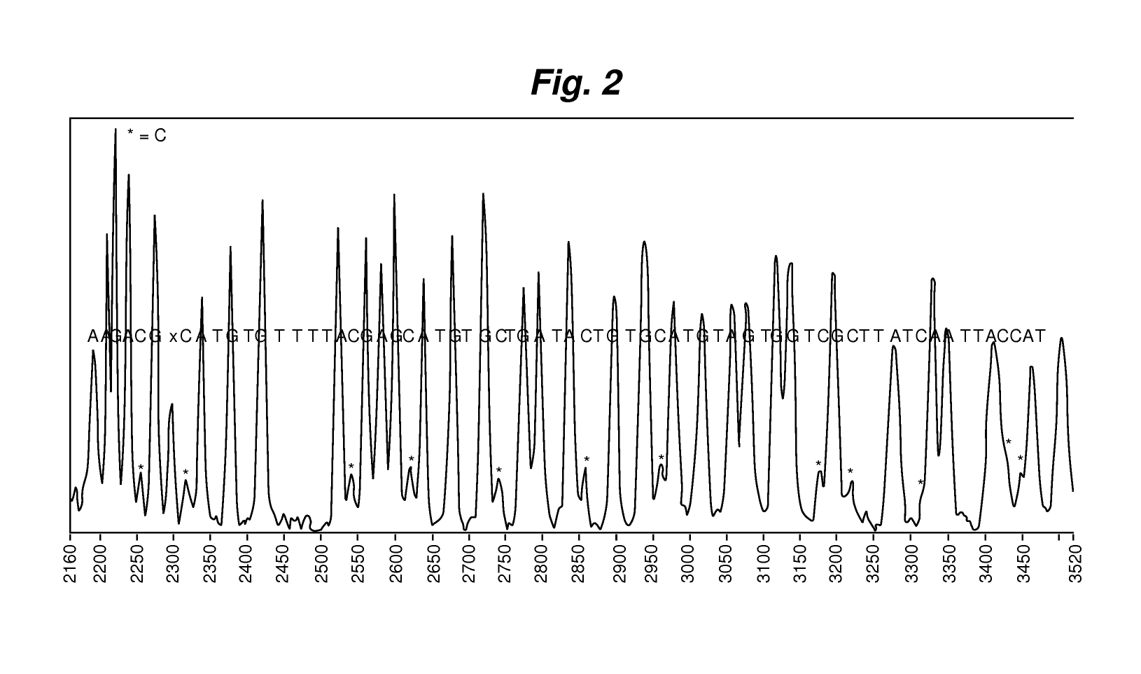

One strand of DNA is labeled at the 5′ position with a fluorescent dye, as indicated with *, in the first sequence (e.g., FAM fluorescence at 540 nm), while the other strand of DNA is labeled at the 3′ position with a different dye which emits at a different wavelength, as indicated with ** (e.g., Cy-5 emitted at 640 nm). A chemical cleavage reaction can be performed for these labeled dsDNA simultaneously with relative efficiency A≈G>C with no significant cleaving efficiency at T positions. After electrophoresis gel separation and fluorescence detection of the cleavage products, the electropherogram that represents the 5′ labeled strand DNA fragments by monitoring the emission at ˜560 nm will show a peak pattern in which peaks with large intensity correspond to nucleotide G and A, peaks with small intensity correspond to nucleotide C, and no peaks correspond to nucleotide T, as shown in FIG. 1. The electropherogram generated by the emission at ˜640 nm will show a peak pattern for the 3′ labeled strand of DNA cleavage fragments, as shown in FIG. 2.

As can be seen in FIGS. 1 and 2, one can not distinguish the G nucleotide from the A nucleotide from any one of strand of DNA cleavage fragment's electropherogram because the cleaving efficiency at nucleotide G and A is similar. However, the C and T positions can be determined because of the relative low abundant signal of C and missing signal of T (shown as a gap). Any small peaks will correspond to the cleavage at C positions while gaps will correspond to the cleavage at T positions.

As shown in Table 1, when one strand has a nucleotide base cleave at A, the complementary strand has a base cleave at T. And, when one strand has a nucleotide base cleave at G, the complementary strand has a base cleave at C. By comparing the peak intensity on both strand DNA fragments electropherograms, the actual nucleotide sequence can be determined.

TABLE 1
Intensity distribution for different base cleavage

| Cleavage | Peak | Cleavage Position | Peak |
|---|---|---|---|
| Position | Intensity | (Complementary Strand) | Intensity |
| A | High | T | No |
| C | Small | G | High |
| G | High | C | Small |
| T | No | A | High |

For example, considering the DNA sequence mentioned previously, and, focusing the cleavage at G and A on the 5′ labeled DNA cleavage fragments, the 5′ labeled DNA strand has the following DNA fragments labeled with detectable dye:

| | |
|---|---|
| *5′-T(T) | no peak (gap) |
| *5′-TT(C) | small peak |
| *5′-TTC(T) | no peak (gap) |
| *5′-TTCT(G) | large peak |
| *5′-TTCTG(C) | small peak |
| *5′-TTCTGC(A) | large peak |
| *5′-TTCTGCA(G) | large peak |
| *5′-TTCTGCAG(T) | no peak (gap) |
| *5′-TTCTGCAGT(A) | large peak |

(G), (I), (A), and (C) correspond to the
cleavage positions.

The complementary strand of DNA has the following fragments corresponding to the 3′ labeled DNA:

| | |
|---|---|
| **3′-A(A) | large peak |
| **3′-AA(G) | large peak |
| **3′-AAG(A) | large peak |
| **3′-AAGA(C) | small peak |
| **3′-AAGAC(G) | large peak |
| **3′-AAGACG(T) | no peak (gap) |
| **3′-AAGACGT(C) | small peak |
| **3′-AAGACGTC(A) | large peak |
| **3′-AAGACGTCA(T) | no peak (gap) |

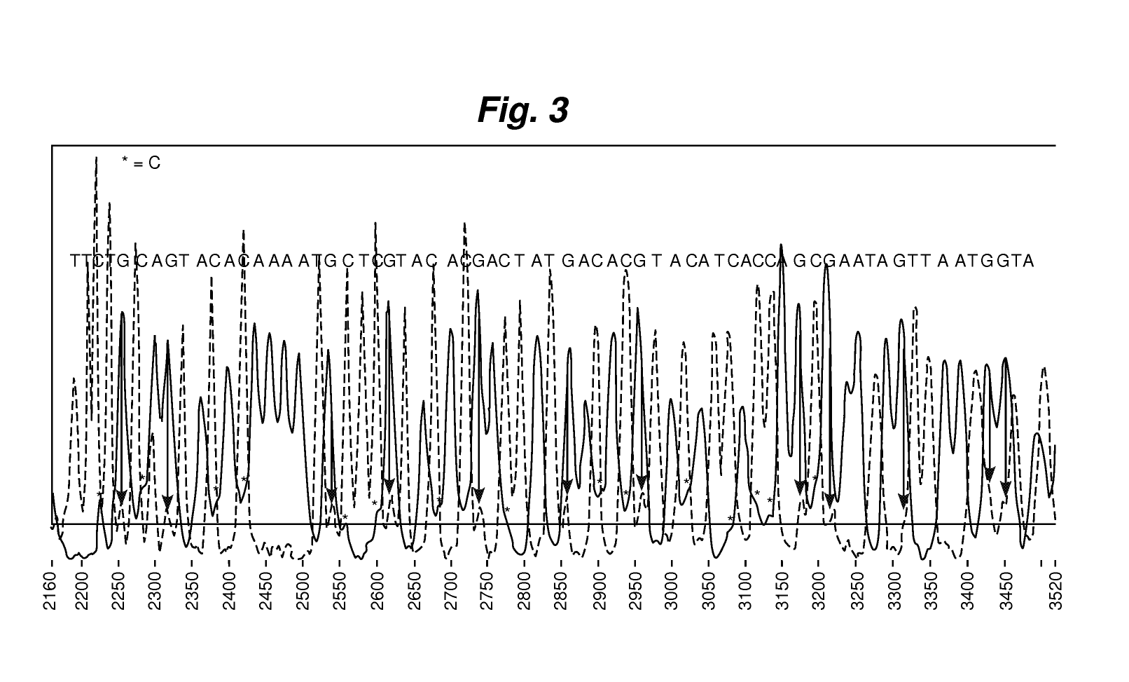

Therefore, any large peak in the 5′ labeled strand DNA electropherogram with a small peak in the 3′ labeled strand DNA electropherogram will indicate the peak as G, while any large peak in the 5′ labeled strand DNA electropherogram with no peak in the 3′ labeled strand DNA electropherogram will indicate the peak as A, as shown in FIG. 3. Therefore, both strands of DNA cleavage information can be used to determine the DNA sequence with one cleaving reaction using a cleaving agent with relative efficiency.

It should be noted that, because T shows as a gap, it may be difficult to determine the exact numbers of Ts if there are several consecutive Ts in sequence in the observed gap. However, the complementary strand of DNA will provide the missing information to complete the sequence determination. When there are multiple Ts in sequence, in which only large spacing is observed in the electropherogram, the other strand's DNA fragments' electropherogram will show multiple peaks that can be used to determine the number of consecutive Ts.

As another example, a chemical cleavage can be performed on G and A with efficiency of G>>A with no cleavage at the C and T positions. In this example, large peaks represent Gs and small peaks represent As. Spacing between peaks represents the location of Cs and Ts. Similar to the previous example, the sequence of G and A can not be determined from any one of the single strand DNA fragments. However, by combining both strands' information, the full DNA sequence can be determined.

Embodiments of the invention also include a DNA nucleotide sequence determined by the use of any of the methods described and/or claimed herein. In some embodiments, the invention includes a composition comprising fragments of dsDNA that has had its respective ssDNA strands differently labeled. These embodiments can be cleaved by a single cleavage reagent having relative cleaving efficiency, or less than four cleaving reagents. Embodiments of the invention also include an electrophoretic gel comprising these dsDNA fragments separated according to size. Other embodiments of the invention include an isolated DNA sequence, or a degenerate variant thereof, comprising a sequence determined by any of the methods described and/or claimed herein. Embodiments of the invention also include a protein or polypeptide sequence corresponding to a nucleotide sequence determined by any of the methods described and/or claimed herein.

Other embodiments of the invention include a computer-readable medium including program instructions for performing the methods described herein. Such a medium can include a magnetic or optical disk or drive, and may be executed by a processor with a user interface. Embodiments of the invention also include a method comprising the use of such a computer-readable medium to perform the methods described and/or claimed herein. In some embodiments, the computer readable medium is programmed to overlay an electropherogram of the first strand with an electropherogram of the second strand to determine the full DNA sequence.

Embodiments of the invention also include a kit consisting of a single, two, or three cleavage reagent(s), together with other ingredients, such as labels (e.g., differentially detectable labels) and instructions for use in performing the methods described and/or claimed herein.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations, which fall within the spirit and broad scope of the invention.

What is claimed is:

1. A method of sequencing DNA, comprising:
separating the strands of a double stranded DNA having complementary nucleotide base pairs, to provide a first single strand and a complementary second single stand;
labeling the first single strand with a first label at its 5′ end;
labeling the second single strand with a second label at its 3′ end, the second label being differentially detectable from the first label;
cleaving the first and second strands with a single cleaving agent having a relative cleaving efficiency;
partially identifying the bases of the first strand;
partially identifying the bases of the second strand; and
determining the sequence of the DNA by combining the partial identification of the first single strand bases and the partial identification of the complementary second single strand bases.

2. The method of claim 1, wherein the relative cleaving efficiency of the cleaving agent is nucleotide 1>nucleotide 2>nucleotide 3>nucleotide 4.

3. The method of claim 1, wherein the relative cleaving efficiency of the cleaving agent is nucleotide 1≈nucleotide 2>nucleotide 3 with insignificant cleaving efficiency for nucleotide 4.

4. The method of claim 1, wherein the relative cleaving efficiency of the cleaving agent is nucleotide 1>nucleotide 2 with insignificant cleaving efficiency for nucleotides 3 and 4.

5. The method of claim 1, wherein the partially identifying steps include an electrophoretic separation.

6. The method of claim 1, wherein the first and second labels are fluorescent dyes and the partially identifying steps include fluorescent emission monitoring.

7. The method of claim 1, wherein the determining the sequence step includes overlaying an electropherogram of the first strand with an electropherogram of the second strand.

8. The method of claim 1, wherein the first and second labels are fluorescent dyes.

9. The method of claim 1, wherein the first label is FAM with fluorescence at 540 nm and the second label is Cy-5 with fluorescence at 640 nm.

10. A method of sequencing DNA, comprising:
separating the strands of a double stranded DNA having complementary nucleotide base pairs, to provide a first single strand and a complementary second single stand;
labeling the first single strand with a first label;
labeling the second single strand with a second label, the second label being differentially detectable from the first label;
cleaving the first and second strands with less than four cleaving agents;
partially identifying the bases of the first strand;
partially identifying the bases of the second strand; and
determining the sequence of the DNA by combining the partial identification of the first single strand bases and the partial identification of the complementary second single strand bases.

11. The method of claim 10, wherein the method consists of cleaving the first and second strands with three cleaving agents.

12. The method of claim 10, wherein the method consists of cleaving the first and second strands with two cleaving agents.

13. The method of claim 10, wherein the method consists of cleaving the first and second strands with a single cleaving agent.


SEQUENCE LISTING (2 sequences)

SEQ ID NO: 1 - DNA, 67 bp, Unknown
  chemically synthesized
ttctgcagtacacaaaatgctcgtacacgactatgacacgtacatcaccagcgaatagttaatggta

SEQ ID NO: 2 - DNA, 67 bp, Unknown
  chemically synthesized
aagacgtcatgtgttttacgagcatgtgctgatactgtgcatgtagtggtcgcttatcaattaccat